SEQUENCE LISTING (8 sequences)

SEQ ID NO: 1 - protein, 118 aa, synthetic construct
  REGION 1..118: note = Synthetic
EVQLLESGGGLVQPGGSLRLSCAASGFTFSDYYMSWIRQAPGKGLEWVSTISGSGGSTYYADSVKGRFTISRDNSKNTLYLQMNSLRAEDTAVYYCARLTAYGHVDSWGQGTLVTVSS

SEQ ID NO: 2 - protein, 112 aa, synthetic construct
  REGION 1..112: note = Synthetic
QSVLTQPPSASGTPGQRVTISCSGSSSNIGTNTVNWYQQLPGTAPKLLIYRNYQRPSGVPDRFSGSKSGTSASLAISGLRSEDEADYYCAAWDDSLSGPHVVFGGGTKLTVL

SEQ ID NO: 3 - protein, 5 aa, synthetic construct
  REGION 1..5: note = Synthetic
DYYMS

SEQ ID NO: 4 - protein, 17 aa, synthetic construct
  REGION 1..17: note = Synthetic
TISGSGGSTYYADSVKG

SEQ ID NO: 5 - protein, 9 aa, synthetic construct
  REGION 1..9: note = Synthetic
LTAYGHVDS

SEQ ID NO: 6 - protein, 13 aa, synthetic construct
  REGION 1..13: note = Synthetic
SGSSSNIGTNTVN

SEQ ID NO: 7 - protein, 7 aa, synthetic construct
  REGION 1..7: note = Synthetic
RNYQRPS

SEQ ID NO: 8 - protein, 13 aa, synthetic construct
  REGION 1..13: note = Synthetic
AAWDDSLSGPHVV

(19) United States
(12) Patent Application Publication
(10) Pub. No.: US 2025/0345333 A1
(43) Pub. Date: Nov. 13, 2025

(54) THERAPIES WITH PPAR AGONISTS AND FGFR4 INHIBITORS

(71) Applicant: TYRA BIOSCIENCES, INC., Carlsbad, CA (US)

(72) Inventors: Ronald SWANSON, Del Mar, CA (US); Todd HARRIS, Carlsbad, CA (US); Mohamed AHMED, Carlsbad, CA (US); Jacqueline STARRETT, Encinitas, CA (US)

(21) Appl. No.: 18/867,134

(22) PCT Filed: May 19, 2023
(86) PCT No.: PCT/US2023/022956

§ 371 Date: Nov. 19, 2024

Related U.S. Application Data
(60) Provisional application No. 63/343,786, filed on May 19, 2022.
(60) Provisional application No. 63/378,098, filed on Oct. 3, 2022.

(51) Int. Cl. A61K 31/517; A61K 31/192; A61K 31/195; A61K 31/216; A61K 31/423; A61K 31/436; A61K 31/496; A61K 31/498; A61K 31/506; A61K 31/519; A61K 31/5377; A61K 31/785; A61K 45/06; A61P 35/00; C07K 16/28

(52) U.S. Cl. CPC A61K 31/517; A61K 31/192; A61K 31/195; A61K 31/216; A61K 31/423; A61K 31/436; A61K 31/496; A61K 31/498; A61K 31/506; A61K 31/519; A61K 31/5377; A61K 31/785; A61K 45/06; A61P 35/00; C07K 16/2863

(57) ABSTRACT

Provided are methods of treating subjects in need of an anti-fibroblast growth factor receptor 4 (anti-FGFR4) therapy. The methods comprise administering to the subjects a therapeutically effective amount of a peroxisome proliferator-activated receptor (PPAR) alpha agonist in combination with the anti-FGFR4 therapy. The methods can comprise administering to the subject a therapeutically effective amount of PPAR alpha agonist and a bile acid sequestrant. Also provided are compositions comprising an FGFR4 inhibitor, a PPAR alpha agonist, and, optionally, a bile acid sequestrant. The methods attenuate the dysregulation of bile acid biosynthesis caused by FGFR4 inhibition with anti-FGFR4 therapies. The methods reduce the severity and/or incidence of adverse events associated with anti-FGFR4 therapies.

### TECHNICAL FIELD

Described herein are methods of treating subjects in need of an anti-fibroblast growth factor receptor 4 (anti-FGFR4) therapy with peroxisome proliferator-activated receptor (PPAR) alpha agonists.

### BACKGROUND

One of the normal physiological roles of FGFR4 is to regulate bile acid synthesis. The FGFR4 stimulation with its ligand, fibroblast growth factor-19 (FGF19), results in the downstream suppression of cholesterol 7α hydroxylase (CYP7A1) expression. CYP7A1 encodes cytochrome P450 7A1 (Cyp7a1 , also known as cholesterol 7-alpha-monooxygenase), which catalyzes the rate limiting step in bile acid synthesis. Inhibition of FGFR4 activity results in an increase in bile acid synthesis due to the lack of repression of CYP7A1. High levels of bile acids can lead to liver toxicity and gastrointestinal issues such as diarrhea. Liver toxicity characterized by single cell necrosis, increased bilirubin, severe diarrhea, and decreased food consumption were reported in monkeys given an anti-FGF19 antibody. In dogs, bile acid sequestration by cholestyramine mitigated FGFR4 inhibition-induced alanine amino transferase (ALT) elevation, an indicator of potential liver toxicity.

FGFR4 inhibitors are under development for treatment of cancers such as hepatocellular carcinoma (HCC). Amplification of the FGFR4 ligand FGF19 and high expression of FGFR4 are frequently reported in HCC cases, and both have been shown to enhance the progression of HCC. FGFR4 has also been implicated in other morbidities, such as in Left Ventricular Hypertrophy (LVH), which is a complication of Chronic Kidney Disease (CKD). Chronic kidney disease is associated with a markedly increased risk of cardiovascular mortality. The dysregulation of bile acids due to FGFR4 inhibition can complicate or even limit therapy.

There remains a need for regulating bile acid synthesis in therapies utilizing FGFR4 inhibitors. The present disclosure addresses these needs.

### SUMMARY

In meeting these needs, the present disclosure provides a method of treating a subject in need of an anti-FGFR4 therapy. The method comprises administering to the subject a therapeutically effective amount of a PPAR alpha agonist in combination with the anti-FGFR4 therapy.

The present disclosure also provides a method of treating a subject in need of an anti-FGFR4 therapy comprising administering to the subject the anti-FGFR4 therapy in combination with a therapeutically effective amount of a PPAR alpha agonist and a bile acid sequestrant.

Also provided are compositions comprising an FGFR4 inhibitor, a PPAR alpha agonist, and a pharmaceutically acceptable excipient.

Also provided are compositions comprising an FGFR4 inhibitor, a PPAR alpha agonist, a bile acid sequestrant, and a pharmaceutically acceptable excipient.

The present disclosure also provides kits comprising an FGFR4 inhibitor and a PPAR alpha agonist.

### BRIEF DESCRIPTION OF THE DRAWINGS

The summary, as well as the following detailed description, is further understood when read in conjunction with the appended drawings. For the purpose of illustrating the disclosed methods, there are shown in the drawings exemplary embodiments of the methods; however, the disclosed methods are not limited to the exemplary embodiments of the methods. In the drawings:

FIGS. 1A and 1B are diagrams depicting the negative feedback mechanism in bile acid (BA) synthesis. In response to an increased BA, enteric hormone FGF19 in humans (FGF15 in mice) is produced and subsequently acts on the liver to inhibit cholesterol 7α hydroxylase (CYP7A1), the rate-limiting enzyme in the classical pathway of BA synthesis, through binding to FGFR4 and subsequent activation of its downstream signaling. FIG. 1C is a diagram depicting the outcome from the use of selective FGFR4 inhibitors: the signaling-induced CYP7A1 downregulation is altered, which results in increased CYP7A1 expression and subsequent increase in BA biosynthesis. FIG. 1D is a diagram depicting inhibition of FGF19 with antibody increases Cyp7a1 and elevates bile acid synthesis resulting in enhanced bile acid efflux and reduced uptake into the hepatocytes. Increased bile acid alters solute transporters in enterocytes and disrupts enterohepatic recirculation of bile acids subsequently causing diarrhea and liver toxicity. Abbreviations: apical sodium-dependent bile acid transporter (ASBT); bile salt export pump (BSEP); ileal bile acid binding protein (IBABP); multidrug resistant protein (MRP) 2, 3, 4; organic anion transporter (OAT); organic solute transporters α-β (OST-α and OST-β); sodium taurocholate cotransporting polypeptide (NTCP). Adapted from Pai et al., Toxicological Sciences 126(2), 446-456 (2012). FIG. 1E is a diagram depicting the effect of PPAR alpha agonists when combined with selective FGFR4 inhibitors: PPAR alpha agonists (e.g., Fibrates) counteract the overexpression of CYP7A1 induced by FGFR4 inhibitors and attenuate the bile acid dysregulation caused by FGFR4 inhibition. FIG. 1F is a schematic diagram depicting the bile acid biosynthetic pathway, the rate limiting enzyme, CYP7A1, and the stable intermediate C4 that is produced upon increased CYP7A1 expression or activity.

FIGS. 2A-2D are bar graphs depicting changes in CYP7A1 expression (Relative mRNA expression/Actin) in Hep3B cells with treatment of increasing concentrations of selective FGFR4 inhibitors fisogatinib (BLU554), roblitinib (FGF-401), erdafitinib (a potent tyrosine kinase inhibitor of FGFR1-4), and futibatinib (a potent and selective covalent inhibitor of FGFR1-4) for 18 hours. Treatment of the cells with BLU554 resulted in increased CYP7A1 expression in Hep3B (FIG. 2A) in a dose dependent manner, as measured using qPCR. Treatment of Hep3B cells with increasing concentrations of BLU-554 (FIG. 2A), FGF-401 (FIG. 2B), erdafitinib (FIG. 2C), futibatinib (FIG. 2D), resulted in an increased CYP7A1 expression (Relative expression/Actin).

FIGS. 3A-3D are bar graphs depicting changes in CYP7A1 expression (Relative mRNA expression/Actin) in HuH-7 cells with treatment of increasing concentrations of selective FGFR4 inhibitors fisogatinib (BLU554), roblitinib (FGF-401), erdafitinib, and futibatinib for 18 hours. The treatment of the cells with the inhibitors resulted in an increased CYP7A1 expression.

FIGS. 4A-4D are bar graphs depicting changes in CYP7A1 expression (Relative mRNA expression/Actin) in Hep3B cells following treatment of the cells with BLU554 (30 nM), FGF-401 (30 nM), erdafitinib (10 nM), or futibatinib (200 nM) in combination with fenofibrate. The treatment resulted in reversal of CYP7A1 expression increase caused by FGFR4 signaling blockade in Hep3B cells.

FIGS. 5A-5D are bar graphs depicting changes in CYP7A1 expression (Relative mRNA expression/Actin) in HuH7 cells following treatment of the cells with BLU554 (30 nM), FGF-401 (30 nM), erdafitinib (10 nM), or futibatinib (200 nM) in combination with fenofibrate. In all cases, except for futibatinib in HuH-7 cells, combination treated with fenofibrate decreased CYP7A1 levels compared to FGFR inhibitor treatment alone.

FIGS. 6A and 6B are bar graphs depicting changes in CYP7A1 expression (Relative expression/Actin) in Hep3B cells co-treated with fenofibric acid and an FGFR4 inhibitor; BLU554 (50 nM, FIG. 6A) or FGF-401 (30 nM, FIG. 6B). Fenofibrate forms an active metabolite, fenofibric acid. Co-treatment of Hep3B cells with fenofibric acid reduced the FGFR4 inhibitor-induced increase in CYP7A1 expression, as shown for both BLU554 (FIG. 6A) and FGF-401 (FIG. 6B).

FIGS. 7A-7D are bar graphs depicting changes in CYP7A1 expression (Relative expression/Actin) in Hep3B cells co-treated with ciprofibrate and an FGFR4 inhibitor BLU-554 (50 nM, FIG. 7A), FGF-401 (30 nM, FIG. 7B), erdafitinib (10 nM, FIG. 7C), and futibatinib (200 nM, FIG. 7D).

FIGS. 8A-8D are bar graphs depicting changes in CYP7A1 expression (Relative expression/Actin) in HuH7 cells co-treated with ciprofibrate and an FGFR4 inhibitor BLU-554 (50 nM, FIG. 8A), FGF-401 (30 nM, FIG. 8B), erdafitinib (10 nM, FIG. 8C), and futibatinib (200 nM, FIG. 8D).

FIGS. 9A-9D are bar graphs depicting changes in CYP7A1 expression (Relative expression/Actin) in Hep3B cells co-treated with gemfibrozil and an FGFR4 inhibitor BLU-554 (50 nM, FIG. 9A), FGF-401 (30 nM, FIG. 9B), erdafitinib (10 nM, FIG. 9C), and futibatinib (200 nM, FIG. 9D).

FIGS. 10A-10D are bar graphs depicting changes in CYP7A1 expression (Relative expression/Actin) in HuH7 cells co-treated with gemfibrozil and an FGFR4 inhibitor BLU-554 (50 nM, FIG. 10A), FGF-401 (30 nM, FIG. 10B), erdafitinib (10 nM, FIG. 10C), and futibatinib (200 nM, FIG. 10D).

FIGS. 11A-11D are bar graphs depicting changes in CYP7A1 expression (Relative expression/Actin) in Hep3B cells co-treated with pemafibrate and an FGFR4 inhibitor BLU-554 (50 nM, FIG. 11A), FGF-401 (30 nM, FIG. 11B), erdafitinib (10 nM, FIG. 11C), and futibatinib (200 nM, FIG. 11D).

FIGS. 12A-12D are bar graphs depicting changes in CYP7A1 expression (Relative expression/Actin) in HuH7 cells co-treated with pemafibrate and an FGFR4 inhibitor BLU-554 (50 nM, FIG. 12A), FGF-401 (30 nM, FIG. 12B), erdafitinib (10 nM, FIG. 12C), and futibatinib (200 nM, FIG. 12D).

FIGS. 13A and 13B are bar graphs depicting increases in levels of serum C4 (ng/ml) in mice treated with anti-FGFR4 therapy, FGF401 at 30 mg/kg or H3B-6527 at 300 mg/kg (FIG. 13A) and BLU554 at 100 mg/kg (FIG. 13B).

FIGS. 14A-14C are bar graphs depicting changes in bile acid (BA) levels (fold change over control) in liver (FIG. 14A), plasma (FIG. 14B), and gallbladder (FIG. 14C) of athymic nude (Nu/Nu) mice treated orally with vehicle ((1) 0.5% MC/tween), H3B-6527 ((2) 300 mg/kg, BID), or FGF401/Roblitinib ((3) 30 mg/kg, BID), for 3 weeks. Four hours after the final dose, mice were euthanized and plasma, liver and gallbladder samples were collected, and bile acid levels measured. The full names of the measured bile acids: LCA: Lithocholic acid; DCA: Deoxycholic acid; TLCA: Taurolithocholic acid; TDCA: Taurodeoxycholic acid; CDCA: Chenodeoxycholic acid; UDCA: ursodeoxycholic acid; CA: Cholic acid; aMCA: α-muricholic acid; bMCA: β-muricholic acid; ωMCA: @-muricholic acid; GCA: Glycodeoxycholic acid; TCDCA: Taurochenodeoxycholic acid; TUDCA: Tauroursodeoxycholic acid; TCA: Taurocholic Acid.

FIGS. 15A and 15B are bar graphs depicting change in serum C4 (ng/ml, FIG. 15A) or Cyp7a1 mRNA expression (% Actb, FIG. 15B) with the treatment of BLU 554 (100 mg/kg) and the indicated doses of fenofibrate (mg/kg). Fenofibrate, administered at indicated concentrations (mg/kg) ameliorated increased serum C4 levels induced by BLU-554 (100 mg/kg) in vivo. FIG. 15B shows that co-administration of fenofibrate with BLU-554 reversed BLU-554-induced Cyp7a1 upregulation in mouse livers, in a dose dependent manner.

FIGS. 16A-16C are graphs depicting changes in serum C4 (ng/ml, FIG. 16A) or tumor volume (mm³, FIGS. 16B and 16C) in athymic nude mice carrying HuH-7 tumors and treated with fenofibrate (40 mg/kg QD), FGF401 (30 or 100 mg/kg BID), BLU554 (100 mg/kg BID (100 mpk)), fenofibrate (40 mg/kg QD) plus FGF401 (30 or 100 mg/kg BID), or fenofibrate (40 mg/kg QD) plus BLU554 (100 mg/kg BID (100 mpk)) for 2 weeks. FIGS. 16B and 16C depict changes in tumor volume (mm³) over time (days) in the mice treated as indicated in FIG. 16A. Data points represent mean tumor volume (n=6 per group) and error bars represent standard error of the mean.

FIGS. 17A-17D are bar graphs depicting CYP7A1 mRNA expression (Relative expression/Actin) in Hep3B cells or HuH-7 cells treated with anti-FGF19 antibody alone or in combination with fenofibrate. Anti-FGF19 antibodies neutralize FGF19 ligand, blocking the signaling pathway of its binding to FGFR4, subsequently activating the downstream signaling that mainly down-regulates CYP7Al expression in hepatocytes. FIGS. 17C and 17D show that fenofibrate can reverse increased CYP7A1 expression induced by FGFR4 inhibition caused by neutralizing FGF19 using anti-FGF19 antibodies. FIG. 17E depicts an exemplary image from a Western blot assay detecting decrease in phosphorylated FGFR4 (Tyr642) following anti-FGF19 antibody treatment.

FIGS. 18A-18B are bar graphs depicting change in CYP7A1 upregulation in Hep3B cells (FIG. 18A), or HuH-7 cells (FIG. 18B), when the cells are treated with the FGFR4 inhibitor FGF401 at 30 nM combined with the indicated concentrations (μM) of PPAR alpha/delta agonist elafibranor. The data depict relative mRNA expression of CYP7A1over Actin.

FIG. 19 is a bar graph depicting changes in serum C4 levels (ng/ml) in C57BL/6 mice dosed orally with vehicle (1), fenofibrate ((2) 100 mg/kg QD), gemfibrozil ((3) 100 mg/kg QD), BLU554 ((4) 100 mg/kg BID), combination BLU554 plus fenofibrate, or combination BLU554 plus gemfibrozil (30 mg/kg QD, 100 mg/kg QD, 150 mg/kg QD, or 300 mg/kg QD) for 6 days. Before the final day of the study, mice were fasted overnight and serum was collected 4 hours after the final dose for C4 analysis. The vehicle for fenofibrate and gemfibrozil was 0.5% methylcellulose/0.5% tween 80, and the vehicle for BLU554 was 80% PEG400/4% hydroxypropyl-B-cyclodextrin. In the combination treatment groups, the compounds were formulated and dosed separately. Columns represent mean C4 value (n=3-4 per group) and error bars represent standard error of the mean.

### DETAILED DESCRIPTION OF ILLUSTRATIVE EMBODIMENTS

The disclosed compositions and methods may be understood more readily by reference to the following detailed description taken in connection with the accompanying figures, which form a part of this disclosure. It is to be understood that the disclosed compositions and methods are not limited to the specific compositions and methods described and/or shown herein, and that the terminology used herein is for the purpose of describing particular embodiments by way of example only and is not intended to be limiting of the claimed compositions and methods.

Unless specifically stated otherwise, any description as to a possible mechanism or mode of action or reason for improvement is meant to be illustrative only, and the disclosed compositions and methods are not to be constrained by the correctness or incorrectness of any such suggested mechanism or mode of action or reason for improvement.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

It is to be appreciated that certain features of the disclosed compositions and methods which are, for clarity, described herein in the context of separate embodiments, may also be provided in combination in a single embodiment. Conversely, various features of the disclosed compositions and methods that are, for brevity, described in the context of a single embodiment, may also be provided separately or in any subcombination.

Various terms relating to aspects of the description are used throughout the specification and claims. Such terms are to be given their ordinary meaning in the art unless otherwise indicated. Other specifically defined terms are to be construed in a manner consistent with the definitions provided herein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. The materials, methods, and examples disclosed herein are illustrative only and not intended to be limiting.

As used herein, the term “substantial” or “substantially” refers to a degree of similarity, difference, increase, or decrease, as in a comparison to a known value. Substantial can include at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% similarity, difference, increase, or decrease, as in a comparison to a known value.

It is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise. The term “about” as used herein when referring to a measurable value such as an amount, a temporal duration, and the like, is meant to encompass variations of ±10%, ±5%, ±1%, or ±0.1% from the specified value, as such variations are appropriate to perform the disclosed methods. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

As used herein, approximating language may be applied to modify any quantitative representation that may vary without resulting in a change in the basic function to which it is related. All ranges are combinable.

Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only.

As used in this specification and the appended claims, the singular forms “a,” “an,” and “the” include plural referents unless the content clearly dictates otherwise. Thus, for example, reference to “a cell” includes a combination of two or more cells, and the like.

As used herein, the terms “individual”, “patient” and “subject”, are used interchangeably to refer to a member of any animal species including, but not limited to, birds, humans and other primates, and other mammals including commercially relevant mammals or animal models such as mice, rats, monkeys, cattle, pigs, horses, sheep, cats, and dogs. Preferably, the subject is a human.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the prevention, management, prophylactic treatment, and/or inhibition or reduction of the number, severity, and/or frequency of one or more symptoms of a disease in a subject. Also as used herein, “treat” and “treatment” may refer to the prevention, management, prophylactic treatment, and/or inhibition or reduction of the number, severity, and/or frequency of one or more adverse events arising from an anti-FGFR4 therapy.

The terms “effective amount” and “therapeutically effective amount” are used interchangeably herein and refer to an amount of the drug effective to achieve a particular biological or therapeutic result such as, but not limited to, amelioration of one or more symptoms of a disease, alleviation of the number, severity, and/or frequency of one or more symptoms of a disease in a subject. The terms “effective amount” and “therapeutically effective amount” are used interchangeably herein and refer to an amount of the drug effective to achieve a particular biological or therapeutic result such as, but not limited to, amelioration of one or more adverse events arising from an anti-FGFR4 therapy. A therapeutically effective amount of drug may vary according to factors such as the disease state, age, sex, body surface area, and body weight of the subject, and the ability of the drug to elicit a desired response in the subject.

As used herein, the term “in need of,” in the context of a subject “in need of” refers to a need for a therapy for treatment of a disease or condition or for the treatment of an adverse event arising from an anti-FGFR therapy.

As used herein, the term “small molecule” refers to a molecule having a molecular weight of less than 1000 gram/mol.

### Combination Therapies

Provided are methods of treating a subject in need of an anti-FGFR4 therapy. The methods comprise administering to the subject a therapeutically effective amount of a PPAR alpha agonist in combination with the anti-FGFR4 therapy. Also provided are methods of treating a subject comprising administering to the subject a therapeutically effective amount of a PPAR alpha agonist in combination with a means for anti-FGFR4 therapy. Means for anti-FGFR4 therapy are known in the art and include, for example, FGFR4 inhibitors.

In some embodiments, the anti-FGFR4 therapy or the means for anti-FGFR4 therapy is an FGFR4 inhibitor. The FGFR4 inhibitor can comprise a direct FGFR4 inhibitor. The direct FGFR4 inhibitor can contact, interact with, bind to, or otherwise alter (e.g., reduce) the level of FGFR4 activity directly. In some embodiments, anti-FGFR4 therapy or the means for an anti-FGFR4 therapy can be a direct FGFR4 inhibitor. Examples of direct FGFR4 inhibitors include small molecule FGFR4 inhibitors, small molecule pan-FGFR inhibitors, or anti-FGFR4 antibodies or binding fragments thereof.

FGFR4 inhibitors can comprise indirect FGFR4 inhibitors. An indirect FGFR4 inhibitor does not contact, interact with, bind to, or otherwise alter (e.g., reduce) the level of FGFR4 activity directly. The indirect FGFR4 inhibitor inhibits the FGFR4 function indirectly, such as by contacting, interacting with, or binding to FGF19, klotho beta (also referred to herein as klotho-β, KLβ, or KLB), or other molecules in the FGFR4 signaling pathway. The indirect FGFR4 inhibitor can comprise an FGFR4 signaling inhibitor. An exemplary FGFR4 signaling inhibitor is an inhibitor that inhibits or reduces the levels of a signaling molecule operating upstream or downstream of FGFR4 in the FGFR4 signaling pathway. In some embodiments, anti-FGFR4 therapy or the means for an anti-FGFR4 therapy can be an indirect FGFR4 inhibitor. Examples of indirect FGFR4 inhibitors include anti-FGF19 antibodies or binding fragments thereof and anti-klotho beta antibodies or binding fragments thereof. Anti-FGF19 antibodies are described at least in U.S. Pat. Nos: 7,678,373; 8,293,241; 8,409,579; and 9,266,955. Anti-klotho beta antibodies are described at least in U.S. Application Publication No: US/2022/0089780. Other examples of anti-klotho beta antibodies or binding fragments thereof include anti-human klotho beta antibodies or their binding fragments obtained from Novus Biologicals (catalog numbers: NBP3-09315; MAB58891; MAB5889; and AF5889), from Affinity Biosciences (catalog number DF14991), from Thermo Fisher Scientific (catalog numbers: PA5-119246 and PA5-44023) or from R&D Systems (catalog numbers: AF2619 and MAB3738).

In some embodiments, the anti-FGFR4 therapy or the means for anti-FGFR4 therapy is a direct FGFR4 inhibitor and/or an indirect FGFR4 inhibitor. The FGFR4 inhibitor can comprise a small molecule FGFR4 inhibitor, or an anti-FGFR4 antibody or a binding fragment thereof. The FGFR4 signaling inhibitor can comprise a small molecule FGFR4 inhibitor, an anti-FGFR4 antibody or a binding fragment thereof, an anti-FGF19 antibody or a binding fragment thereof, or an anti-klotho beta antibody or a binding fragment thereof. The small molecule FGFR4 inhibitor includes, but is not limited to, roblitinib (FGF401), H3B-6527, ICP-105, fisogatinib (BLU554), INCB062079, erdafitinib, futibatinib, pemigatinib, infigratinib, and a combination thereof.

The FGFR4 inhibitor can comprise an anti-FGFR4 antibody or a binding fragment thereof. The anti-FGFR4 antibody can comprise the U3-1784 antibody or the binding fragment thereof. The U3-1784 antibody or the binding fragment thereof comprises a heavy chain variable region and a light chain variable region of the following amino acid sequences (Bartz et al., Mol Cancer Ther 2019;18:1832-43; the complementarity determining regions (CDRs) in the heavy chain variable region and the light chain variable region are underlined):

Heavy chain variable region
SEQ ID NO: 1
EVQLLESGGGLVQPGGSLRLSCAASGFTFSDYYMSWIRQAPGKGLEWV
STISGSGGSTYYADSVKGRFTISRDNSKNTLYLQMNSLRAEDTAVYYCA
RLTAYGHVDSWGQGTLVTVSS
Light chain variable region
SEQ ID NO: 2
QSVLTQPPSASGTPGQRVTISCSGSSSNIGTNTVNWYQQLPGTAPKLLI
YRNYQRPSGVPDRFSGSKSGTSASLAISGLRSEDEADYYCAAWDDSLSG
PHVVFGGGTKLTVL

The FGFR4 signaling inhibitor can comprise an anti-FGF19 antibody or a binding fragment thereof. The anti-FGF19 antibody can comprise an FGF19 neutralizing antibody.

The anti-FGFR4 therapy or means for anti-FGFR4 therapy can comprise a combination of an FGFR4 inhibitor and a second chemotherapeutic agent. In some embodiments, the second chemotherapeutic agent is an immune checkpoint inhibitor. In some embodiments, the immune checkpoint inhibitor is an antibody or a binding fragment of an antibody. The immune checkpoint inhibitor can comprise the antibody or the binding fragment of an antibody that binds programmed death-1 (PD1), programmed death ligand-1 (PD-L1), programmed death ligand-2 (PD-L2), or cytotoxic T-lymphocyte-associated antigen 4 (CTLA4).

In some embodiments, the PPAR-alpha agonist is a small molecule. In some embodiments, the PPAR-alpha agonist is fenofibrate, fenofibric acid, ciprofibrate, gemfibrozil, bezafibrate, elafibranor, pemafibrate, or a combination thereof. Fenofibrate is a prodrug, which after absorption is hydrolyzed by tissue and plasma esterases to its principal active metabolite fenofibric acid. Elafibranor is a dual PPARα/δ agonist. The chemical structures of fenofibrate, fenofibric acid,, ciprofibrate, gemfibrozil, bezafibrate, elafibranor, and pemafibrate are shown below:

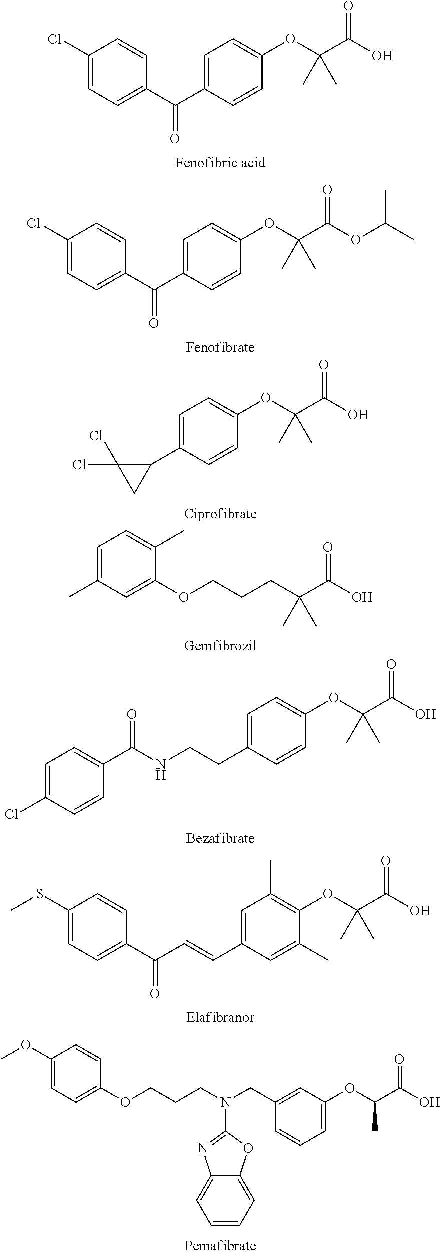

The disclosed methods can comprise administering the FGFR4 inhibitor at amount of between about 0.5 mg and about 3000 mg daily, in one or more doses. For example, the disclosed methods can comprise administering the FGFR4 inhibitor at an amount between about 0.5 mg and about 3000 mg, about 1 mg and about 2500 mg, about 5 mg and about 2000 mg, about 10 mg and about 3000 mg, about 15 mg and about 3000 mg, about 20 mg and about 3000 mg, about 25 mg and about 3000 mg, about 30 mg and about 3000 mg, about 35 mg and about 3000 mg, about 50 mg and about 3000 mg, about 45 mg and about 3000 mg, or about 50 mg and about 3000 mg, daily, in one or more doses. The disclosed methods can comprise administering the FGFR4 inhibitor at an amount between about 0.5 mg and about 2500 mg, about 1 mg and about 2000 mg, about 5 mg and about 1500 mg, about 10 mg and about 1000 mg, about 15 mg and about 500 mg, about 20 mg and about 250 mg, about 25 mg and about 200 mg, about 30 mg and about 150 mg, about 35 mg and about 100 mg, about 40 mg and about 100 mg, about 45 mg and about 100 mg, or about 50 mg and about 100 mg, daily, in one or more doses.

The disclosed methods can comprise administering the FGFR4 inhibitor at an amount of between about 0.01 mg/kg and about 50 mg/kg daily. The FGFR4 inhibitor can be administered in one or more doses. For example, the disclosed methods can comprise administering the FGFR4 inhibitor at an amount between about 0.01 mg/kg and about 50 mg/kg, about 0.05 mg/kg and about 50 mg/kg, about 0.1 mg/kg and about 50 mg/kg, about 0.5 mg/kg and about 50 mg/kg, about 1 mg/kg and about 50 mg/kg, about 5 mg/kg and about 50 mg/kg, about 10 mg/kg and about 50 mg/kg, about 15 mg/kg and about 50 mg/kg, about20 mg/kg and about 50 mg/kg, about 25 mg/kg and about 50 mg/kg, about 30 mg/kg and about 50 mg/kg, about 35 mg/kg and about 50 mg/kg, about 40 mg/kg and about 50 mg/kg, or about 45 mg/kg and about 50 mg/kg, daily, in one or more doses.

In some aspects, the disclosed methods comprise administering the FGFR4 inhibitor orally to the subject in a fed or a fasted state.

In some aspects, the disclosed methods comprise administering the FGFR4 inhibitor by injection. In some aspects, the disclosed methods comprise administering the FGFR4 inhibitor by intravenous injection.

The disclosed methods can comprise administering the PPAR alpha agonist at an amount between about 0.05 mg and about 3000 mg daily in one or more doses. For example, the disclosed methods can comprise administering the PPAR alpha agonist at an amount between about 0.05 mg and about 3000 mg, about 0.1 mg and about 3000 mg, about 1 mg and about 2500 mg, about 5 mg and about 2000 mg, about 10 mg and about 3000 mg, about 15 mg and about 3000 mg, about 20 mg and about 3000 mg, about 25 mg and about 3000 mg, about 30 mg and about 3000 mg, about 35 mg and about 3000 mg, about 50 mg and about 3000 mg, about 45 mg and about 3000 mg, or about 50 mg and about 3000 mg, daily, in one or more doses. The disclosed methods can comprise administering the PPAR alpha agonist at an amount between about 0.05 mg and about 2500 mg, about 0.1 mg and about 2000 mg, about 1 mg and about 2000 mg, about 5 mg and about 1500 mg, about 10 mg and about 1000 mg, about 15 mg and about 500 mg, about 20 mg and about 250 mg, about 25 mg and about 200 mg, about 30 mg and about 150 mg, about 35 mg and about 100 mg, about 40 mg and about 100 mg, about 45 mg and about 100 mg, or about 50 mg and about 100 mg, daily, in one or more doses . . .

The disclosed methods can comprise administering the PPAR alpha agonist at an amount between about 0.001 mg/kg and about 50 mg/kg daily. For example, the disclosed methods can comprise administering the PPAR alpha agonist at an amount between about 0.001 mg/kg and about 50 mg/kg, about 0.005 mg/kg and about 50 mg/kg, about 0.01 mg/kg and about 50 mg/kg, about 0.05 mg/kg and about 50 mg/kg, about 0.1 mg/kg and about 50 mg/kg, about 0.5 mg/kg and about 50 mg/kg, about 1 mg/kg and about 50 mg/kg, about 5 mg/kg and about 50 mg/kg, about 10 mg/kg and about 50 mg/kg, about 15 mg/kg and about 50 mg/kg, about20 mg/kg and about 50 mg/kg, about 25 mg/kg and about 50 mg/kg, about 30 mg/kg and about 50 mg/kg, about 35 mg/kg and about 50 mg/kg, about 40 mg/kg and about 50 mg/kg, or about 45 mg/kg and about 50 mg/kg, daily, in one or more doses.

In some embodiments, the methods further comprising administering to the subject a bile acid sequestrant. In some embodiments, the bile acid sequestrant is cholestyramine, colestipol, colesevelam, or a combination thereof. In some embodiments, the bile acid sequestrant is cholestyramine.

The disclosed methods can comprise administering the PPAR alpha agonist prior to, simultaneously with, or following administration of the anti-FGFR4 therapy. In those methods wherein a bile acid sequestrant is further administered, the bile acid sequestrant can be administered prior to, simultaneously with, or following administration of the PPAR alpha agonist. In those methods wherein a bile acid sequestrant is further administered, the bile acid sequestrant can be administered prior to, simultaneously with, or following administration of the anti-FGFR4 therapy.

Also disclosed are methods treating a subject in need of an anti-FGFR4 therapy comprising administering to the subject the anti-FGFR4 therapy in combination with a therapeutically effective amount of a PPAR alpha agonist and a bile acid sequestrant. The disclosed methods can comprise administering the PPAR alpha agonist and the bile acid sequestrant prior to, simultaneously with, or following administration of the anti-FGFR4 therapy.

Examples of combination therapies are presented in Tables 1 and 2.

TABLE 1
Representative combination therapies with small
molecule FGFR4 inhibitor and PPAR alpha agonist.

| anti-FGFR4 therapy | | | | | |
|---|---|---|---|---|---|
| (0.01-50 mg/kg) | | | | | |
| Small molecule | PPAR alpha agonist (0.001-50 mg/kg) | | | | |
| FGFR4 inhibitor | fenofibrate | fenofibric acid | ciprofibrate | gemfibrozil | bezafibrate |
| roblitinib (FGF401) | + | +/− | +/− | +/− | +/− |
| H3B-6527 | + | +/− | +/− | +/− | +/− |
| ICP-105 | + | +/− | +/− | +/− | +/− |
| fisogatinib (BLU554) | + | +/− | +/− | +/− | +/− |
| INCB062079 | + | +/− | +/− | +/− | +/− |
| roblitinib (FGF401) | +/− | + | +/− | +/− | +/− |
| H3B-6527 | +/− | + | +/− | +/− | +/− |
| ICP-105 | +/− | + | +/− | +/− | +/− |
| fisogatinib (BLU554) | +/− | + | +/− | +/− | +/− |
| INCB062079 | +/− | + | +/− | +/− | +/− |
| roblitinib (FGF401) | +/− | +/− | + | +/− | +/− |
| H3B-6527 | +/− | +/− | + | +/− | +/− |
| ICP-105 | +/− | +/− | + | +/− | +/− |
| fisogatinib (BLU554) | +/− | +/− | + | +/− | +/− |
| INCB062079 | +/− | +/− | + | +/− | +/− |
| roblitinib (FGF401) | +/− | +/− | +/− | + | +/− |
| H3B-6527 | +/− | +/− | +/− | + | +/− |
| ICP-105 | +/− | +/− | +/− | + | +/− |
| fisogatinib (BLU554) | +/− | +/− | +/− | + | +/− |
| INCB062079 | +/− | +/− | +/− | + | +/− |
| roblitinib (FGF401) | +/− | +/− | +/− | +/− | + |
| H3B-6527 | +/− | +/− | +/− | +/− | + |
| ICP-105 | +/− | +/− | +/− | +/− | + |
| fisogatinib (BLU554) | +/− | +/− | +/− | +/− | + |
| INCB062079 | +/− | +/− | +/− | +/− | + |

“+” indicates a combination therapy of the small molecule FGFR4 inhibitor and the PPAR alpha agonist
“+/−” indicates an inclusion (+) or exclusion (−) of the specific PPAR alpha agonist in the combination therapy of the small molecule FGFR4 inhibitor and the PPAR alpha agonist

TABLE 2
Combination therapies with a small molecule FGFR4 inhibitor,
PPAR alpha agonist, and a bile acid sequestrant.

anti-FGFR4

| therapy | | | | | | Sequestrant |
|---|---|---|---|---|---|---|
| (0.01-50 mg/kg) | PPAR alpha agonist (0.001-50 mg/kg) | | | | | cholestyramine |
| Small molecule | | Fenofibric | | | | colestipol and/ |
| FGFR4 inhibitor | Fenofibrate | acid | Ciprofibrate | Gemfibrozil | Bezafibrate | or colesevelam |
| roblitinib (FGF401) | + | +/− | +/− | +/− | +/− | + |
| H3B-6527 | + | +/− | +/− | +/− | +/− | + |
| ICP-105 | + | +/− | +/− | +/− | +/− | + |
| fisogatinib (BLU554) | + | +/− | +/− | +/− | +/− | + |
| INCB062079 | + | +/− | +/− | +/− | +/− | + |
| roblitinib (FGF401) | +/− | + | +/− | +/− | +/− | + |
| H3B-6527 | +/− | + | +/− | +/− | +/− | + |
| ICP-105 | +/− | + | +/− | +/− | +/− | + |
| fisogatinib (BLU554) | +/− | + | +/− | +/− | +/− | + |
| INCB062079 | +/− | + | +/− | +/− | +/− | + |
| roblitinib (FGF401) | +/− | +/− | + | +/− | +/− | + |
| H3B-6527 | +/− | +/− | + | +/− | +/− | + |
| ICP-105 | +/− | +/− | + | +/− | +/− | + |
| fisogatinib (BLU554) | +/− | +/− | + | +/− | +/− | + |
| INCB062079 | +/− | +/− | + | +/− | +/− | + |
| roblitinib (FGF401) | +/− | +/− | +/− | + | +/− | + |
| H3B-6527 | +/− | +/− | +/− | + | +/− | + |
| ICP-105 | +/− | +/− | +/− | + | +/− | + |
| fisogatinib (BLU554) | +/− | +/− | +/− | + | +/− | + |
| INCB062079 | +/− | +/− | +/− | + | +/− | + |
| roblitinib (FGF401) | +/− | +/− | +/− | +/− | + | + |
| H3B-6527 | +/− | +/− | +/− | +/− | + | + |
| ICP-105 | +/− | +/− | +/− | +/− | + | + |
| fisogatinib (BLU554) | +/− | +/− | +/− | +/− | + | + |
| INCB062079 | +/− | +/− | +/− | +/− | + | + |

+ indicates a combination therapy of the small molecule FGFR4 inhibitor, the PPAR alpha agonist, and a bile acid sequestrant
+/− indicates an inclusion (+) or exclusion (−) of the specific PPAR alpha agonist in the combination therapy of the small molecule FGFR4 inhibitor, the PPAR alpha agonist, anda bile acid sequestrant

The disclosed methods are administered to a subject in need of treatment for proliferative disease, metabolic disease, cardiovascular disease, or kidney disease. The subject can be in need of treatment for a proliferative disease that is an FGFR4-mediated cancer, hepatocellular carcinoma, cholangiocarcinoma, or a solid tumor. The subject can be in need of treatment for a metabolic disease, for example non-alcoholic steatohepatitis (NASH) or diabetes. The subject can be in need of treatment for type 2 diabetes. The subject can be in need of treatment for concentric cardiac hypertrophy. The subject can be in need of treatment for cardiovascular disease. The subject can be in need of treatment for chronic kidney disease. The subject can be in need of treatment for left ventricular hypertrophy.

The disclosed methods can provide one or more of: a reduction in the number of anti-FGFR4 therapy-related adverse events, a reduction in the anti-FGFR4 therapy-related adverse event frequency, a reduction in the anti-FGFR4 therapy-related adverse event severity, an increase in duration of the anti-FGFR4 therapy, an increase in daily dose of the anti-FGFR4 therapy, and an increase in patient compliance of the subject to the anti-FGFR4 therapy. In some embodiments, the adverse event is diarrhea, nausea, vomiting, increased level of aspartate transaminase (AST), increased level of alanine transaminase (ALT), increased level of gamma-glutamyl transferase (GGT), increased level of serum bilirubin, increased prothrombin time (PT), or a combination thereof.

In some embodiments, the methods provide a reduction in serum levels of C4 (7-alpha-hydroxy-4-cholestene-3-one), a bile acid, or a combination thereof. The methods can provide a reduction in serum level of C4 by between about 5% and about 95% in the subject as compared to that in a subject receiving the anti-FGFR4 therapy without the PPAR alpha agonist. For example, the methods can provide a reduction in serum level of C4 by between about 5% and about 10%, between about 5% and about 15%, between about 5% and about 20%, between about 5% and about 25%, between about 5% and about 30%, between about 5% and about 35%, between about 5% and about 40%, between about 5% and about 45%, between about 5% and about 50%, between 5% and about 55%, between about 5% and about 60%, between about 5% and about 65%, between about 5% and about 70%, between about 5% and about 75%, between about 5% and about 80%, between about 5% and about 85%, between about 5% and about 90%, or between about 5% and about 95% in the subject as compared to the serum level of C4 in a subject receiving the anti-FGFR4 therapy without the PPAR alpha agonist.

### Compositions

Also disclosed are compositions comprising an FGFR4 inhibitor, a PPAR alpha agonist, and a pharmaceutically acceptable excipient. The compositions can comprise the FGFR4 inhibitor and the PPAR alpha agonist as a single unit dosage form.

Also disclosed are compositions comprising an FGFR4 inhibitor, a PPAR alpha agonist, a bile acid sequestrant, and a pharmaceutically acceptable excipient. The disclosed compositions can comprise the FGFR4 inhibitor, the PPAR alpha agonist, the bile acid sequestrant, and the pharmaceutically acceptable excipient in a single unit dosage form.

In some embodiments, the FGFR4 inhibitor in the compositions is a small molecule FGFR4 inhibitor. In some embodiments, the small molecule FGFR4 inhibitor is roblitinib (FGF401), H3B-6527, ICP-105, fisogatinib (BLU554), INCB062079, erdafitinib, futibatinib, pemigatinib, infigratinib, or a combination thereof.

In some embodiments, the FGFR4 inhibitor in the compositions is an anti-FGFR4antibody or a binding fragment thereof, or an anti-FGR19 antibody or a binding fragment thereof. In some embodiments, the anti-FGFR4 antibody is U3-1784 or the binding fragment thereof.

In some embodiments, the PPAR alpha agonist in the compositions is fenofibrate, fenofibric acid, ciprofibrate, gemfibrozil, bezafibrate, elafibranor, pemafibrate, or a combination thereof. In some embodiments, the bile acid sequestrant is cholestyramine, colestipol, colesevelam, or a combination thereof.

### Kits

Also provided are kits comprising an FGFR4 inhibitor and a PPAR alpha agonist. The kits can comprise the FGFR4 inhibitor as a single unit dosage form and the PPAR alpha as a single unit dosage form. The kits can comprise the FGFR4 inhibitor and the PPAR alpha on the same blister pack. The kits can further comprise a bile acid sequestrant.

The kits can comprise instructions for use and a dosing chart with dosing and regimen recommendations.

### EXAMPLES

FGFR4 inhibitors are under development for treatment of cancers such as hepatocellular carcinoma (HCC), treatment of solid tumors, CKD, and cardiovascular conditions. The dysregulation of bile acids due to FGFR4 inhibition can complicate or even limit the anti-FGFR4 therapy in subjects.

### Example 1. FGFR4 Inhibitors Increase CYP7A1 Expression and BA Biosynthesis, Which is Attenuated by the PPAR Alpha Agonists Fenofibrate and Fenofibric Acid

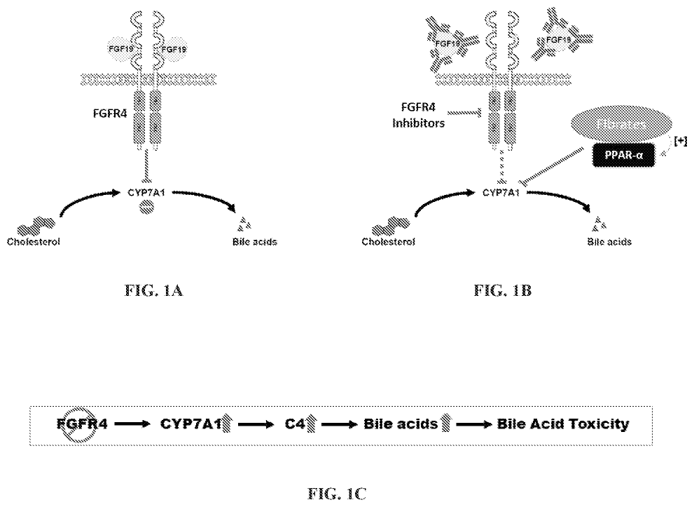

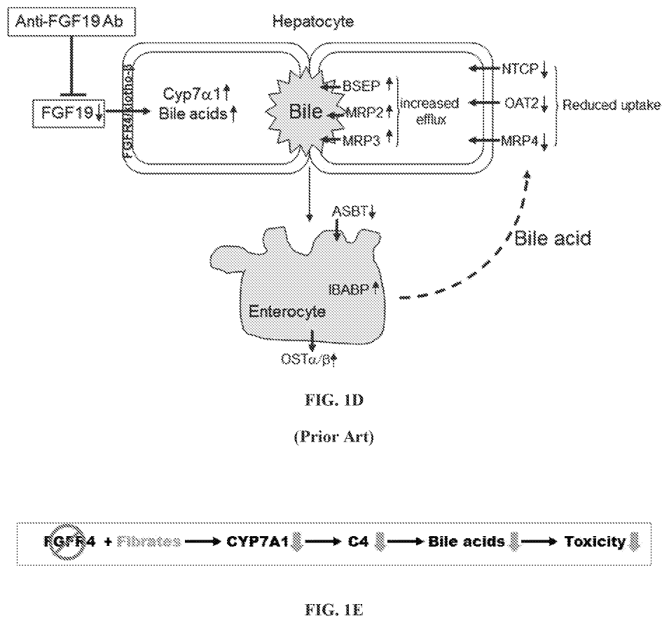

An exemplary negative feedback mechanism for bile acid (BA) synthesis is depicted in FIGS. 1A and 1B. Selective inhibition of FGFR4 with inhibitors increases BA biosynthesis (FIGS. 1C and 1D). The PPAR alpha agonists counteract this increase (FIG. 1E).

### Materials and Methods

The examples detail that the use of FGFR4 inhibitors with or without PPAR alpha agonists demonstrated that PPAR alpha agonists attenuated the BA dysregulation caused by FGFR4 inhibitors in in vitro cell studies and in vivo treatments.

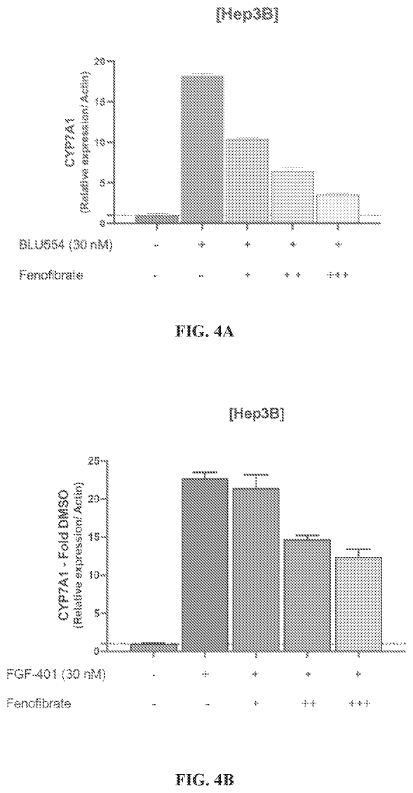

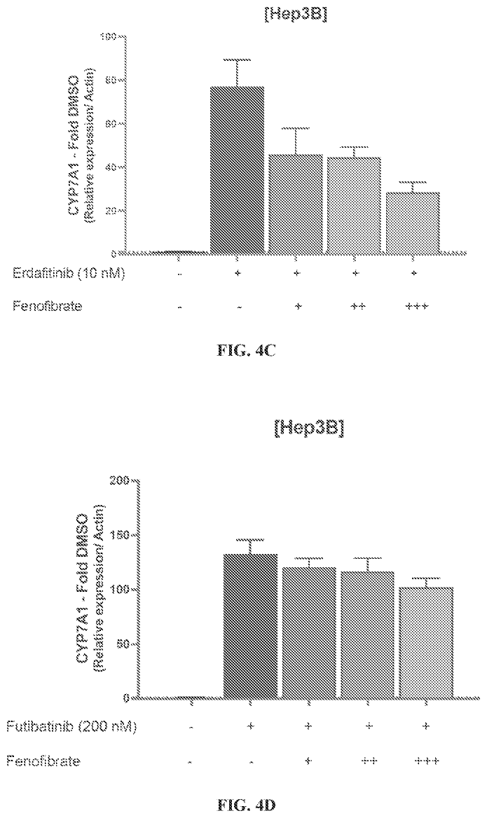

Treatment of HCC cell lines (Hep3B and HuH-7) with the selective FGFR4 inhibitors BLU-554 (30 nM) or FGF401 (30 nM), or pan-FGFR inhibitors erdafitinib (10 nM) or futibatinib (200 nM), in combination with increasing concentrations of fenofibrate or fenofibric acid (2, 5, or 10 μM) resulted in reversal of the CYP7A1 upregulation that was induced by FGFR4 signaling inhibition (FIGS. 4A-6B).

For the in vivo experiments, male C57BL/6 mice were dosed orally with vehicle, fenofibrate, an FGFR4 inhibitor, or with a combination of fenofibrate and an FGFR4 inhibitor at the doses indicated. Before the final day of the study, animals were fasted overnight and serum was collected 4 hours after the final dose. Serum C4 (7-alpha-hydroxy- 4-cholestene-3-one) analyses were carried out on an Agilent 6495 triple quadrupole mass spectrometer with jet stream source coupled to an Agilent 1290 UPLC stack. Data was processed using Waters TargetLynx data processing software. Calibration curves were generated using reference standard (7A4C; Sigma-Aldrich). Calibration range was from 0.5ng/ml-200 ng/ml with an internal deuterated standard (7A4C-D7; Avanti Polar Lipids) concentration of 100 ng/ml. The vehicle for fenofibrate, FGF401 and H3B-6527 was 0.5% methylcellulose/0.5% tween 80 (MC/Tween), and the vehicle for BLU554 was 80% PEG400/4% hydroxypropyl-β-cyclodextrin (HPBCD). When combination treatments were tested, the vehicle treatment groups received both vehicles, formulated separately. In the combination treatment groups, the compounds were formulated and dosed separately. The mean C4 value for each group of 4 mice is shown, and error bars represent standard error of the mean.

### Results

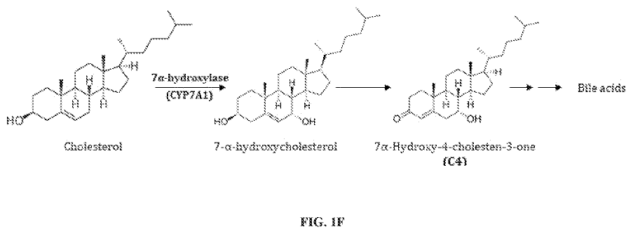

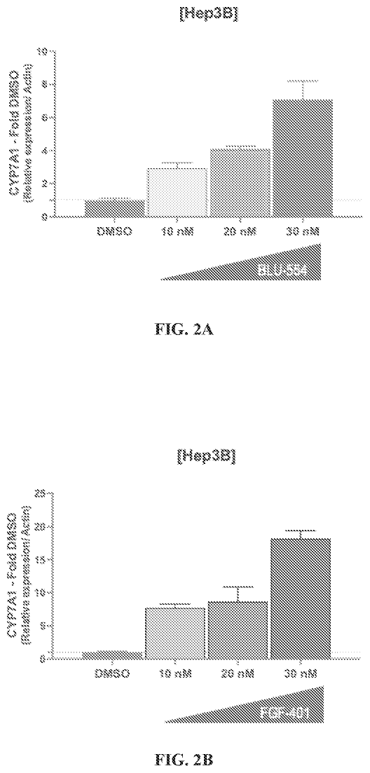

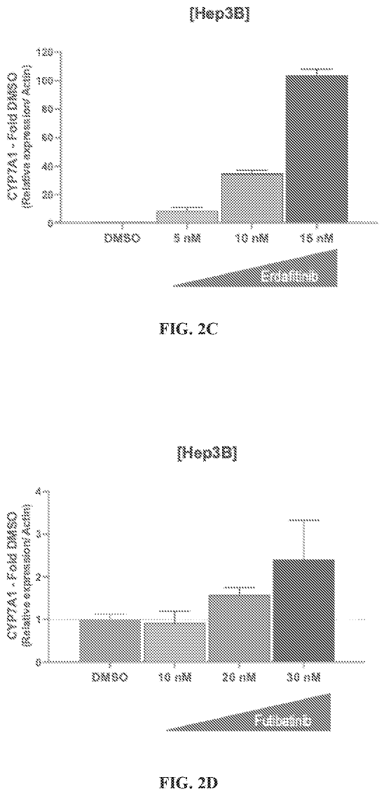

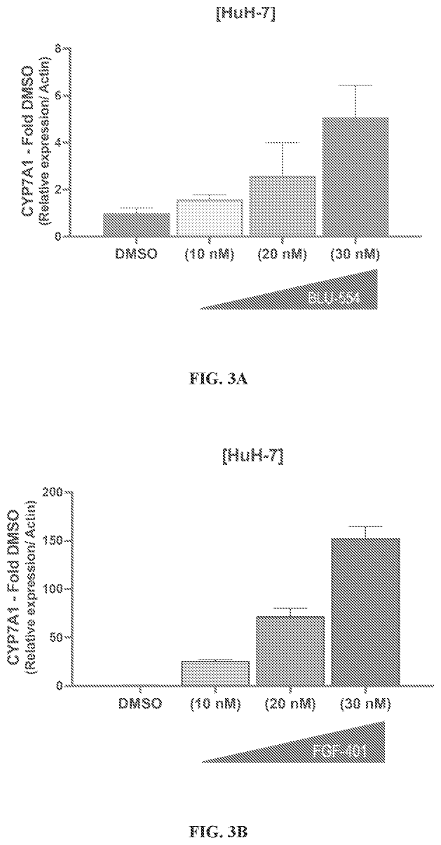

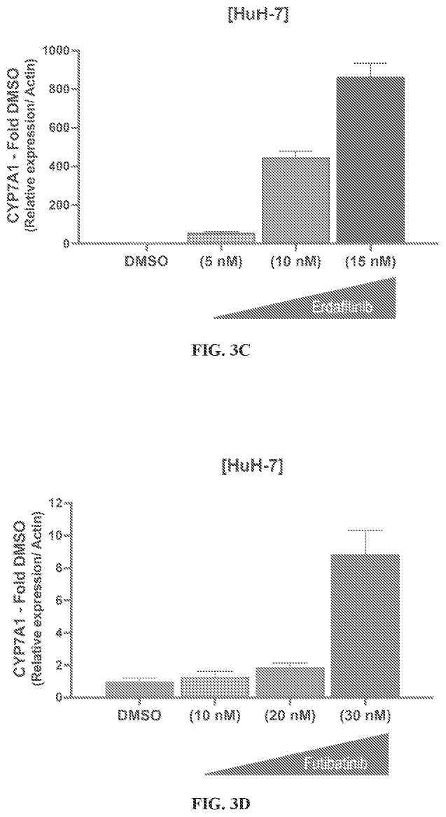

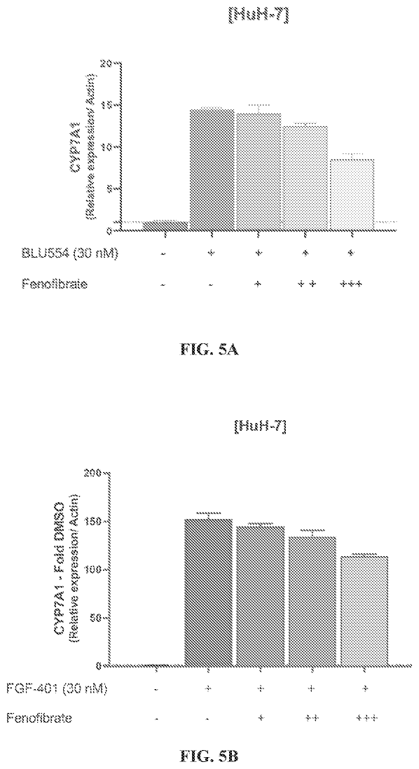

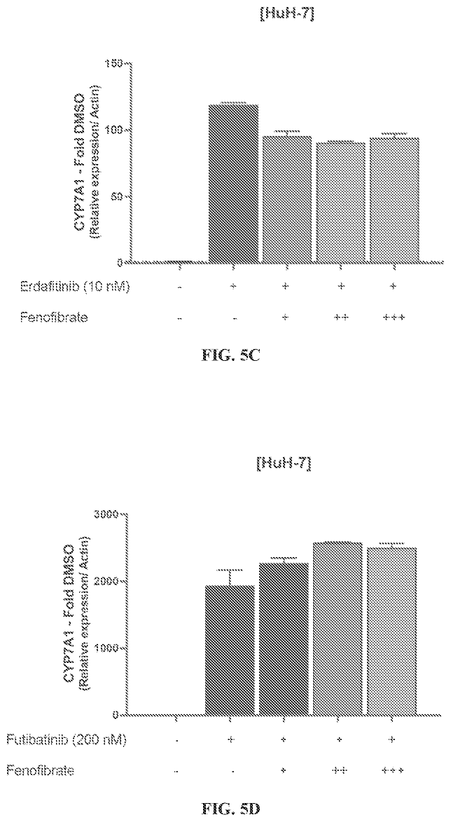

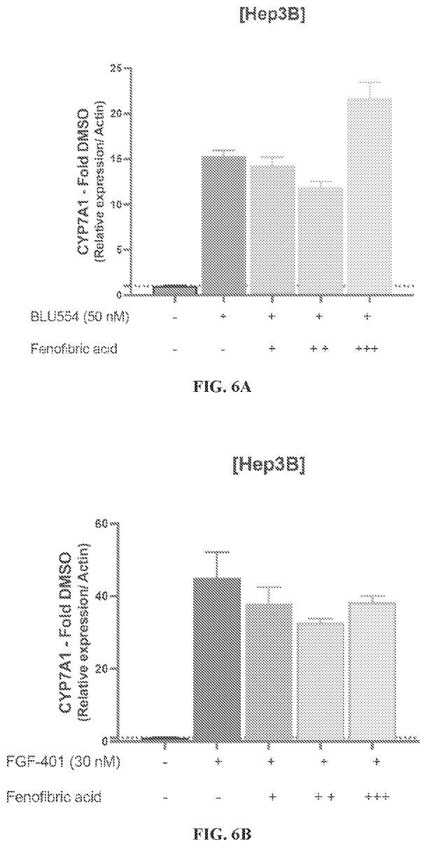

As shown in FIGS. 2A-2D and 3A-3D, the treatment of cells with increasing concentrations of selective FGFR4 inhibitors for 18 hours resulted in increase in CYP7A1 expression (Relative expression/Actin) in 2 hepatocellular carcinoma (HCC) cell lines: Hep3B cells (FIGS. 2A-2D) and HuH-7 cells (FIGS. 3A-3D). Treatment of the cells with FGFR4 specific inhibitors BLU-554 and FGF-401 and with pan-FGFR inhibitors erdafitinib and futibatinib resulted in increased CYP7A1 expression in Hep3B (FIGS. 2A-2D) and HuH-7 cells (FIGS. 3A-3D) in a dose dependent manner, as measured using qPCR. Treatment of the cells with BLU-554 (30 nM) in combination with Fenofibrate resulted in reversal of CYP7A1 expression increase caused by FGFR4 signaling blockade using BLU-554 in both Hep3B (FIG. 4A) and HuH-7 cells (FIG. 5A). Treatment of the cells with FGFR4-specific inhibitor FGF401 (FIGS. 4B and 5B) and pan-FGFR inhibitors erdafitinib (FIGS. 4C and 5C) and futibatinib (FIGS. 4D and 5D) in combination with fenofibrate also resulted in reversal of the increase in CYP7A1 expression, except in the case of futibatinib in HuH-7 cells. Hep3B cells were also treated with fenofibric acid, the active metabolite of fenofibrate. Co-treatment of BLU554 (FIG. 6A) or FGF401 (FIG. 6B) treated cells with fenofibric acid also reduced CYP7A1 levels. Fenofibrate or fenofibric acid was applied at a concentration of 2, 5, and 10 μM added simultaneously in combination with 30 nM of BLU-554 in growth media (DMEM supplemented with 10% Fetal Bovine Serum), for 18 hours. mRNA was then isolated and CYP7A1 expression was measured using TaqMan™ qPCR Assays (CYP7A1 assay ID: Hs00167982_m1, ACTNB assay ID: Hs99999903_m1).

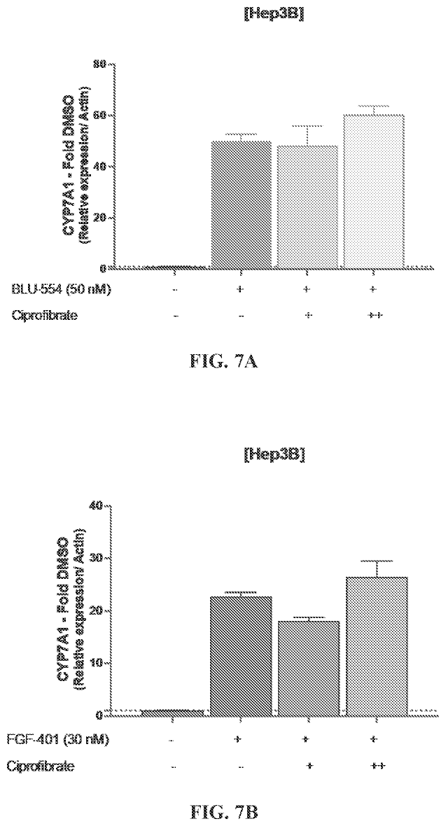

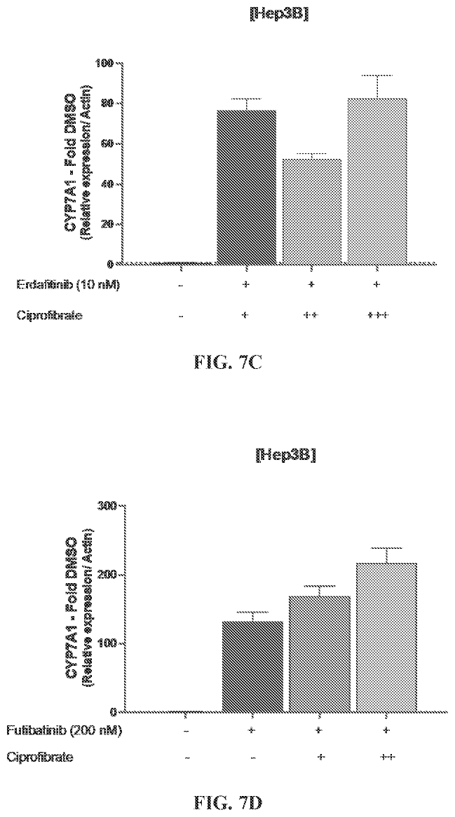

FIGS. 7A-7D are bar graphs depicting changes in CYP7A1 expression (Relative expression/Actin) in Hep3B cells co-treated with ciprofibrate and an FGFR4 inhibitor BLU-554 (50 nM, FIG. 7A), FGF-401 (30 nM, FIG. 7B), erdafitinib (10 nM, FIG. 7C), and futibatinib (200 nM, FIG. 7D).

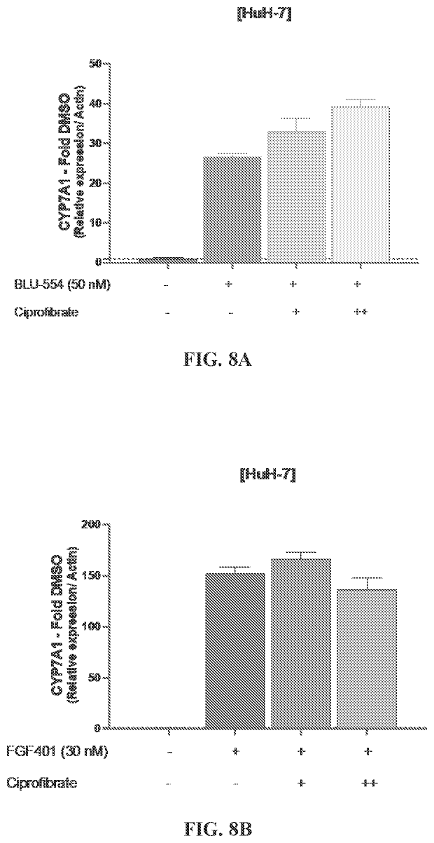

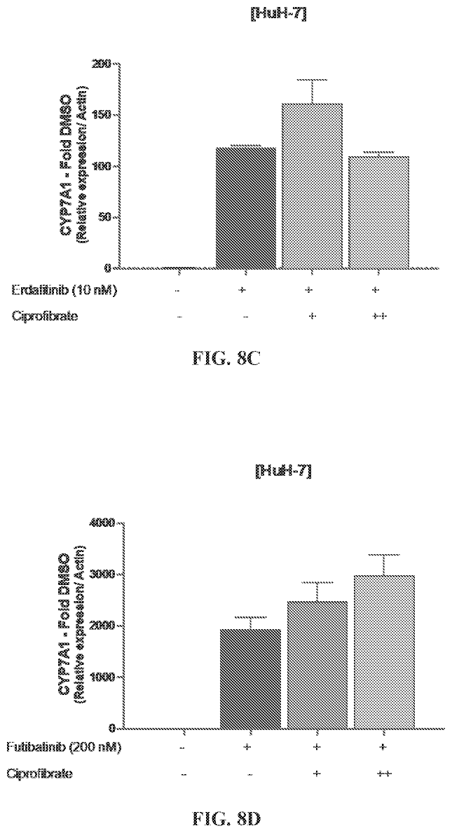

FIGS. 8A-8D are bar graphs depicting changes in CYP7A1 expression (Relative expression/Actin) in HuH7 cells co-treated with ciprofibrate and an FGFR4 inhibitor BLU-554 (50 nM, FIG. 8A), FGF-401 (30 nM, FIG. 8B), erdafitinib (10 nM, FIG. 8C), and futibatinib (200 nM, FIG. 8D).

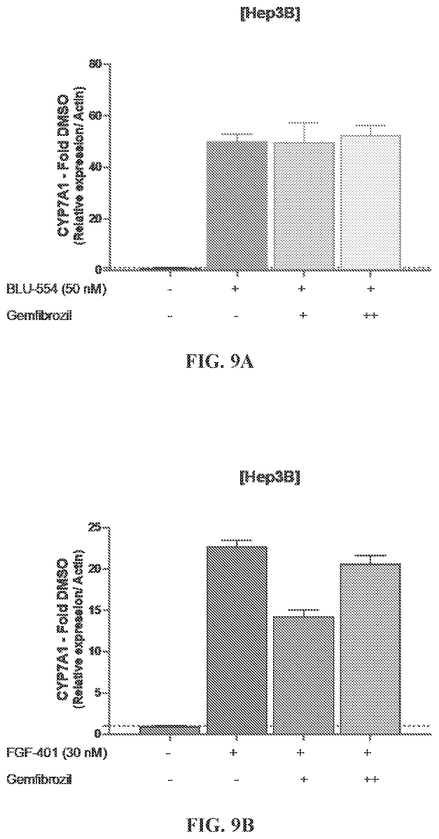

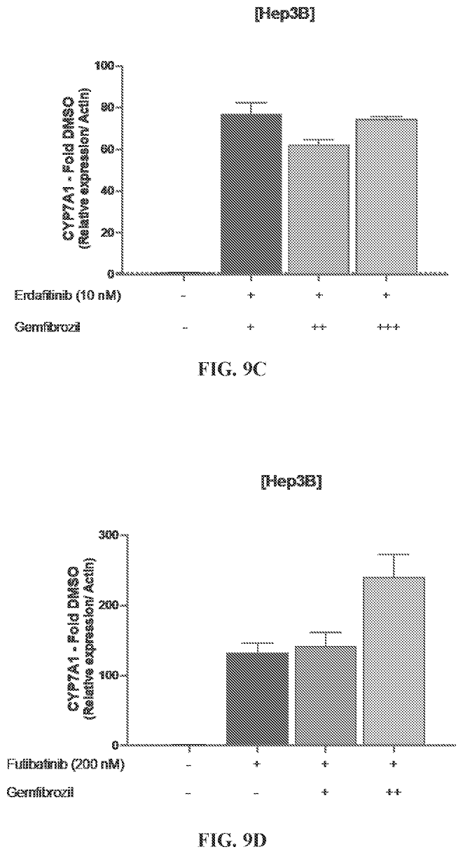

FIGS. 9A-9D are bar graphs depicting changes in CYP7A1 expression (Relative expression/Actin) in Hep3B cells co-treated with gemfibrozil and an FGFR4 inhibitor BLU-554 (50 nM, FIG. 9A), FGF-401 (30 nM, FIG. 9B), erdafitinib (10 nM, FIG. 9C), and futibatinib (200 nM, FIG. 9D).

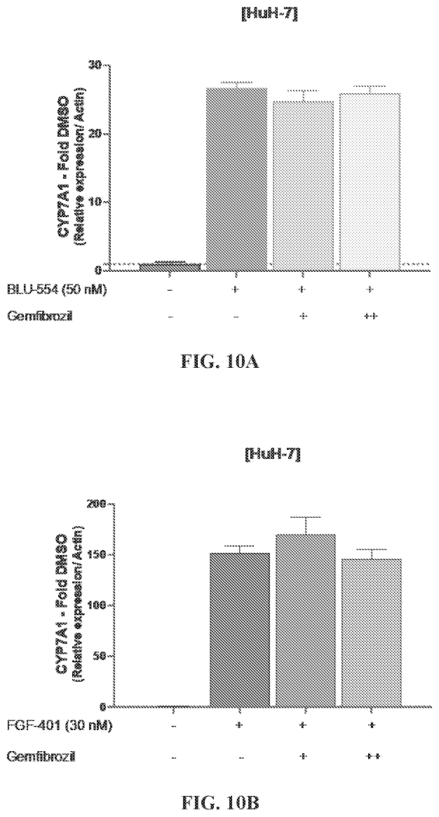

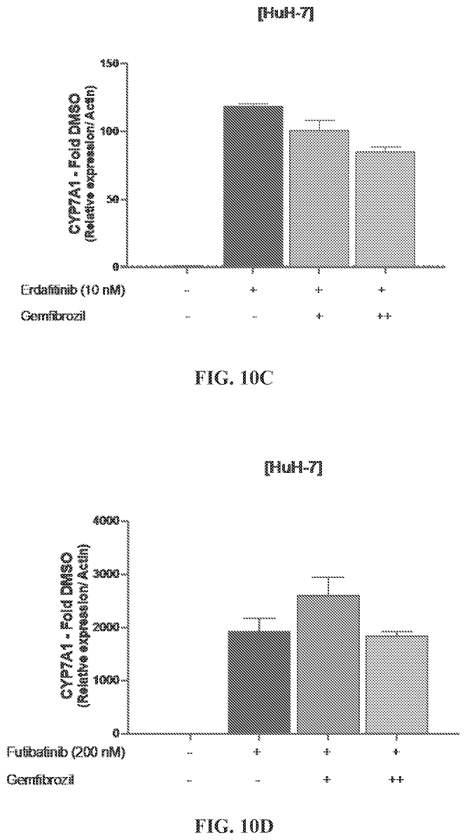

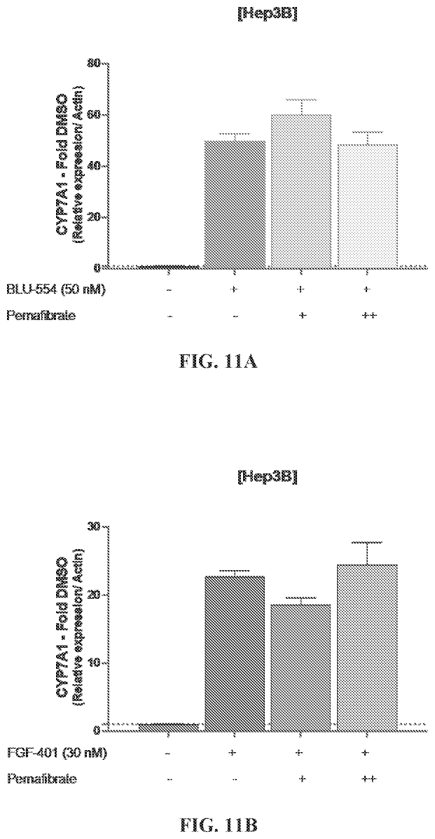

FIGS. 10A-10D are bar graphs depicting changes in CYP7A1 expression (Relative expression/Actin) in HuH7 cells co-treated with gemfibrozil and an FGFR4 inhibitor BLU-554 (50 nM, FIG. 10A), FGF-401 (30 nM, FIG. 10B), erdafitinib (10 nM, FIG. 10C), and futibatinib (200 nM, FIG. 10D).

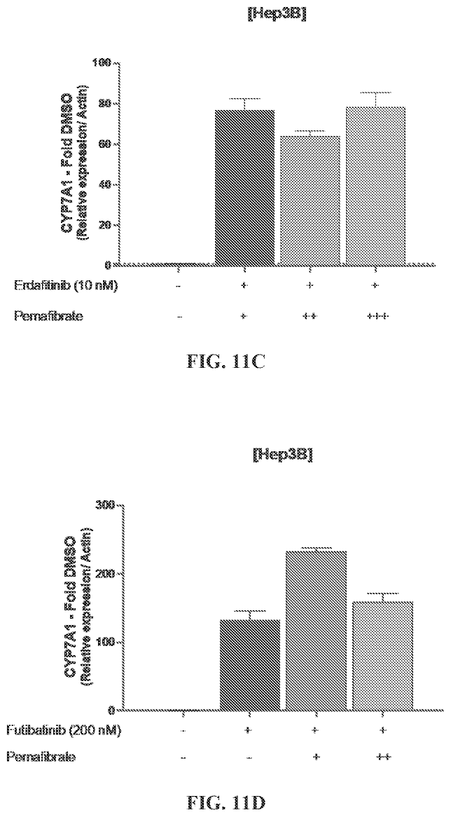

FIGS. 11A-11D are bar graphs depicting changes in CYP7A1 expression (Relative expression/Actin) in Hep3B cells co-treated with pemafibrate and an FGFR4 inhibitor BLU-554 (50 nM, FIG. 11A), FGF-401 (30 nM, FIG. 11B), erdafitinib (10 nM, FIG. 11C), and futibatinib (200 nM, FIG. 11D).

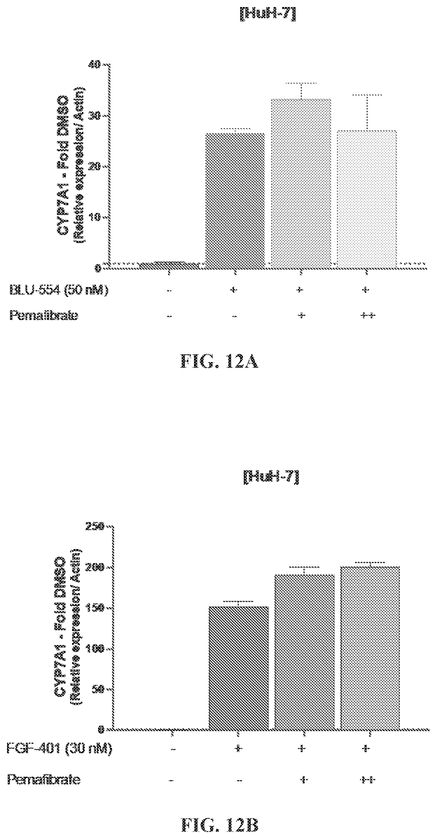

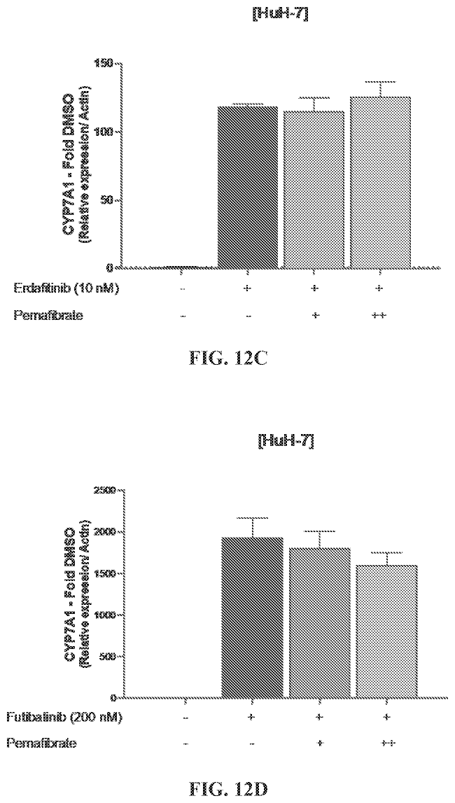

FIGS. 12A-12D are bar graphs depicting changes in CYP7A1 expression (Relative expression/Actin) in HuH7 cells co-treated with pemafibrate and an FGFR4 inhibitor BLU-554 (50 nM, FIG. 12A), FGF-401 (30 nM, FIG. 12B), erdafitinib (10 nM, FIG. 12C), and futibatinib (200 nM, FIG. 12D).

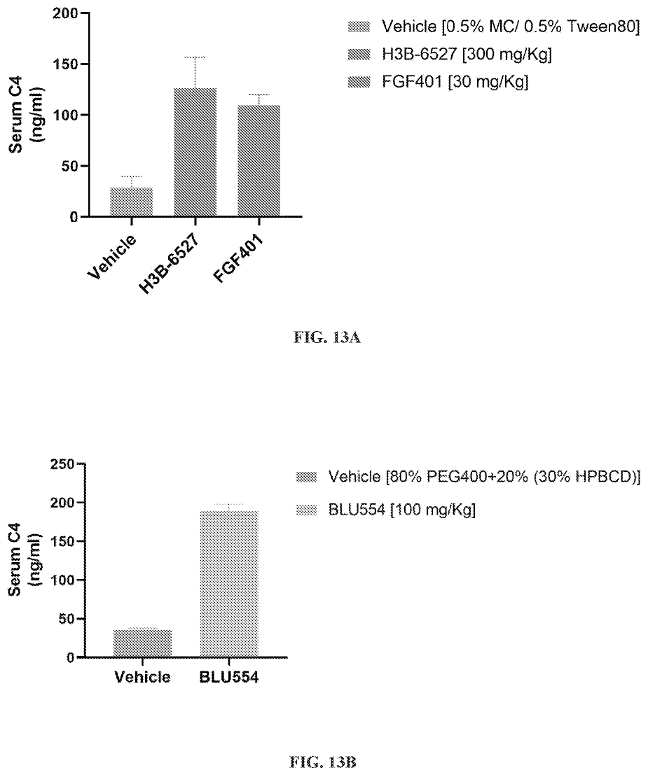

FIGS. 13A and 13B are bar graphs demonstrating changes in serum C4 levels (ng/ml) in mice treated with anti-FGFR4 therapy; athymic nude (Nu/Nu) mice were treated with H3B-6527 (300 mg/Kg) or FGF401 (30 mg/Kg) (FIG. 13A), and C57BL/6 mice were treated with BLU-554 (100 mg/Kg) (FIG. 13B).

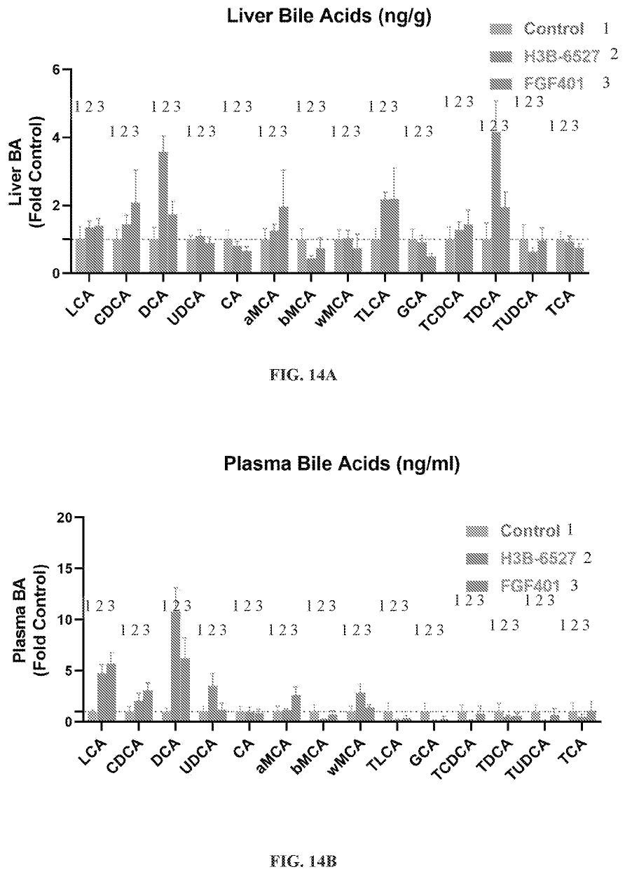

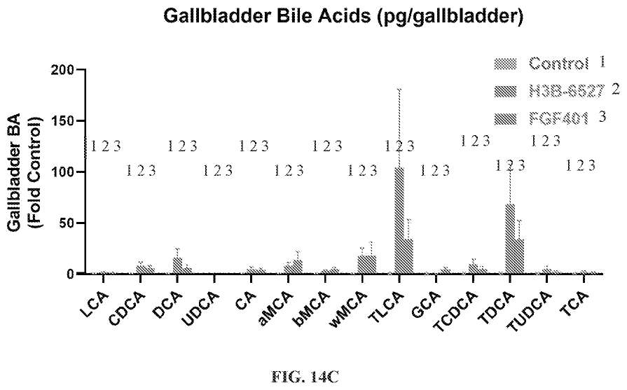

FIGS. 14A-14C are bar graphs depicting changes of bile acid (BA) levels (fold change over control) in liver (FIG. 14A), plasma (FIG. 14B), and gallbladder (FIG. 14C) of athymic nude (Nu/Nu) mice treated orally with vehicle (0.5% MC/tween), H3B-6527 (300 mg/kg, BID), or FGF401/Roblitinib (30 mg/kg, BID), for 3 weeks. Four hours after the final dose, mice were euthanized and plasma, liver and gallbladder samples were collected and bile acid levels measured. Bile acids were measured at the metabolomics core facility of University of Kansas Medical Center, following an established protocol. Treatment with either H3B-6527 or FGF-401 resulted in increased bile acids level as measure in plasma, liver, and gall bladder, when compared to vehicle (control) treated mice.

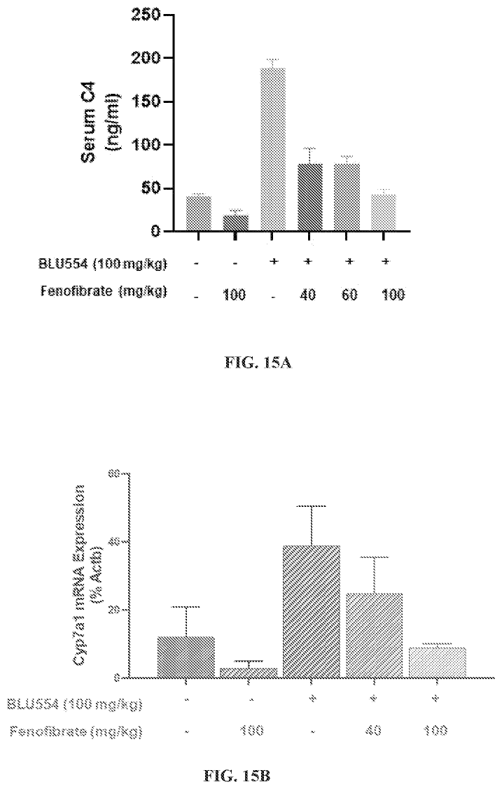

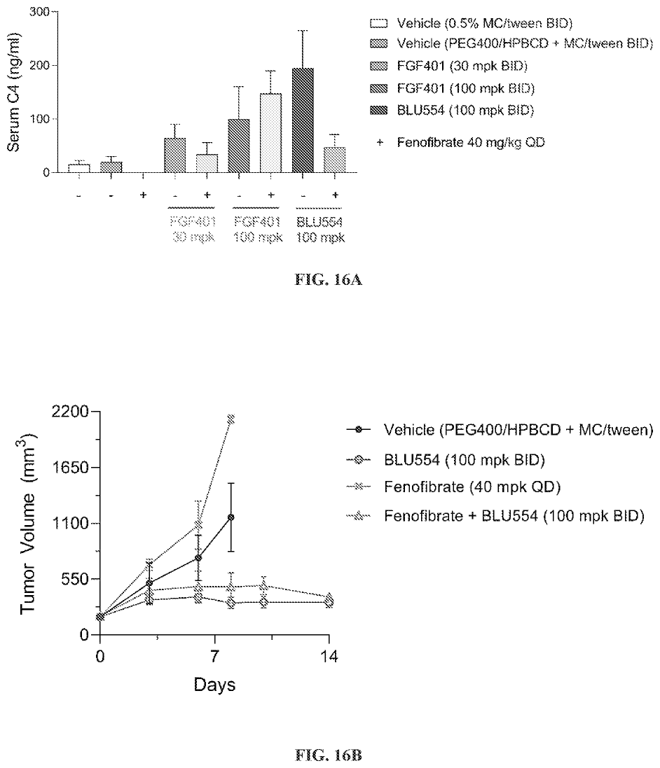

FIG. 15A demonstrates changes in serum C4 levels in mice treated with with BLU-554 (100 mg/Kg) alone or in combination with different doses of PPAR alpha agonist Fenofibrate for 6 days, showing that fenofibrate reduced the increase in C4 caused by BLU554. The data in FIG. 15B depict a change in liver Cyp7a1 mRNA expression (% Actb) with the different tested conditions, which correlate with the serum C4 levels in FIG. 15A.

### Example 2. Fenofibrate Attenuates FGFR4 Inhibitor-Induced C4 Increase in Tumor-Bearing Mice

### Materials and Methods

The compound BLU554, a covalent highly specific FGFR4 inhibitor, was tested in a HuH-7 xenograft mouse model. HuH-7 is a human hepatocellular carcinoma cell line driven by FGFR4 by virtue of an FGF19 amplification.

Athymic nude Nu/Nu mice were injected subcutaneously in the flank with a human hepatocellular carcinoma cell line, HuH-7, which has an amplification of FGF19. Once the tumors were about 150 mm³, mice were dosed orally with vehicle, fenofibrate (40 mg/kg QD), FGF401 (30 or 100 mg/kg BID), BLU554 (100 mg/kg BID), fenofibrate (40 mg/kg QD) plus FGF401 (30 or 100 mg/kg BID), or fenofibrate (40 mg/kg QD) plus BLU554 (100 mg/kg BID) for 14 days. Animals were fasted overnight, and serum was collected 4 hours after the final dose. Serum C4 (7-alpha-hydroxy-4-cholestene-3-one) analyses were carried out on an Agilent 6495 triple quadrupole mass spectrometer with jet stream source coupled to an Agilent 1290 UPLC stack. Data was processed using Waters TargetLynx data processing software. Calibration curves were generated using reference standard (7A4C; Sigma-Aldrich). Calibration range was from 0.5ng/ml-200 ng/ml with an internal deuterated standard (7A4C-D7; Avanti Polar Lipids) concentration of 100 ng/ml. The vehicle for fenofibrate and FGF401 was 0.5% methylcellulose/0.5% tween 80, and the vehicle for BLU554 was 80% PEG400/4% hydroxypropyl-β-cyclodextrin. The vehicle treatment group received both vehicles, formulated separately. In the combination treatment group, the compounds were formulated and dosed separately. The mean C4 value for each group of 6 mice is shown, and error bars represent standard error of the mean.

### Results

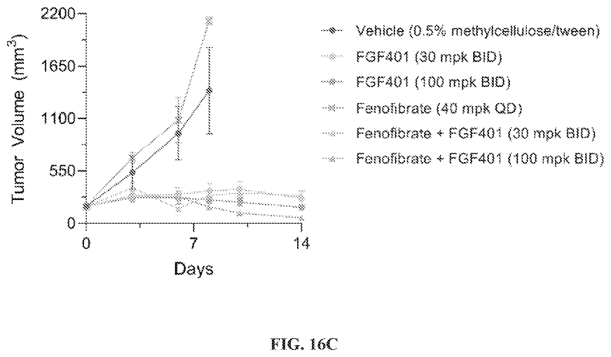

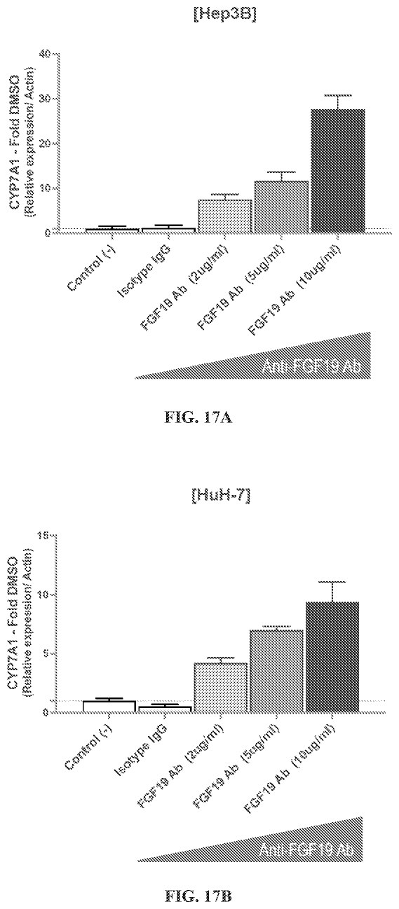

As shown in FIG. 16A, BLU554 and FGF401 treatment alone significantly raised levels of 7 alpha-hydroxy-4-cholesten-3-one (C4), a peripheral marker of Cyp7a1 activity and served as an indirect measurement of hepatic bile acid synthesis. By contrast, co-treatment of BLU554 treated animals with fenofibrate, a PPAR alpha agonist, significantly attenuated the rise in C4. Co-treatment of FGF401 treated animals with fenofibrate also attenuated the rise in C4, but only at the 30 mg/kg BID dose, not the 100 mg/kg BID dose. These results show a method of limiting the toxicity induced by FGFR4 inhibitors, by co-treatment or co-administration of FGFR4 inhibitors with a PPAR alpha agonist. FIGS. 16B-16C are graphs depicting changes in tumor volume (mm³) over time (days) in the mice treated as indicated in FIG. 16A. Data points represent mean tumor volume (n=6 per group) and error bars represent standard error of the mean.

### Example 3. Fenofibrate Ameliorates Neutralizing Anti-FGF19 Antibody-Induced CYP7A1 Upregulation in FGFR4-Dependent/FGF19-Amplified Hepatocellular Carcinoma Cell Lines

### Materials and Methods

Hep3B and HUH-7 cells were treated with 10 μg/ml mouse Isotype monoclonal IgG control (R&D-MAB002), or 2 μg/ml, 5 μg/ml, and 10 μg/ml of hFGF19 Antibody (R&D-AF969) for 24 hours. mRNA was then isolated and CYP7A1 expression was measured using qPCR. The depicted data represent the fold change in CYP7A1 mRNA levels compared to vehicle (DMSO) treated cells, and Actin was used as internal control.

Hep3B and HUH-7 cells were treated with 10 μg/ml of hFGF19 Antibody (R&D-AF969) in combination with 2 μM, 5 μM, or 10 μM of fenofibrate, for 24 hours. Two control groups included; untreated cells, and cell treated with 10 μg/ml mouse Isotype monoclonal IgG (R&D-MAB002), for 24 hours.

### Results

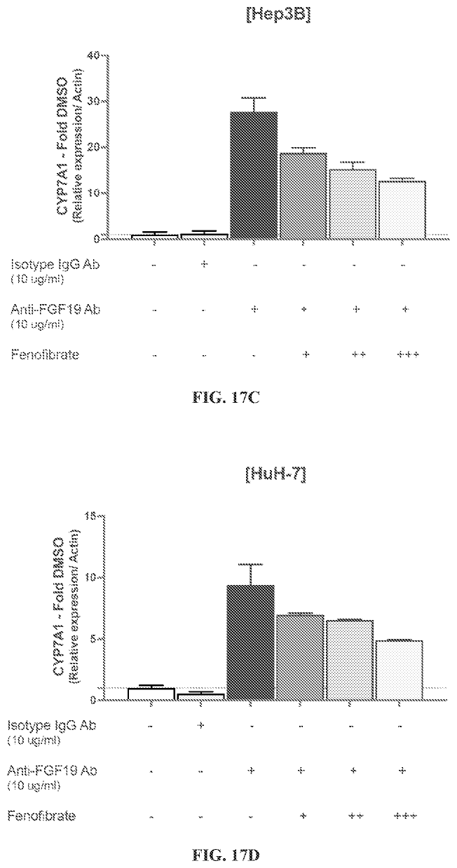

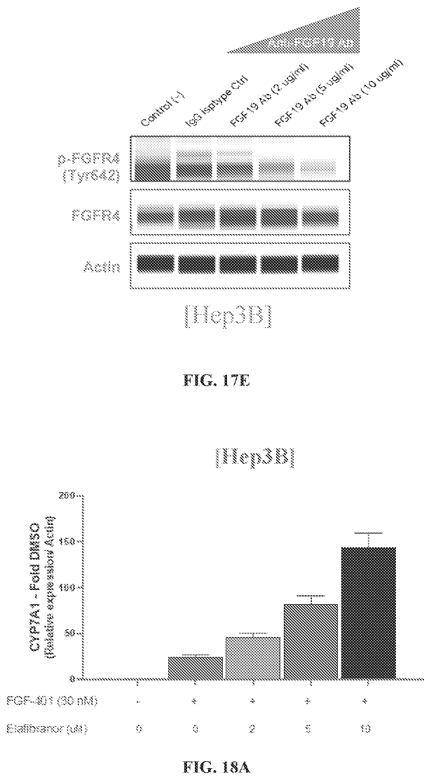

Treatment of the cells with neutralizing anti-FGF19 antibody resulted in increased CYP7A1 expression in Hep3B (FIG. 17A) and HuH-7 cells (FIG. 17B) in a dose dependent manner. Treatment of the cells with neutralizing anti-FGF19 (10 μg/ml) in combination with fenofibrate resulted in reversal of CYP7A1 expression increase caused by FGFR4 signaling blockade using neutralizing anti-FGF19 antibody in both Hep3B (FIG. 17C) and HuH-7 cells (FIG. 17D).

### Example 4. Effect of PPAR Agonist Elafibranor on the CYP7A1 Expression Induced by FGF401

FIGS. 18A-18B are bar graphs depicting change in CYP7A1 upregulation in Hep3B cells (FIGS. 18A), or HuH-7 cells (FIGS. 18B), when the cells are treated with the FGFR4 inhibitor FGF401 at 30 nM and with the indicated concentrations (μM) of PPAR alpha/delta agonist elafibranor. The data depict relative expression of CYP7A1 over Actin.

Elafibranor did not attenuate the increase in in CYP7A1 upregulation in this assay when tested at the indicated concentrations.

### Example 5. PPAR Agonists Gemfibrozil and its Effect on Serum C4 In Vivo

C57BL/6 mice were dosed orally with vehicle, fenofibrate (100 mg/kg QD), gemfibrozil (100 mg/kg QD), BLU554 (100 mg/kg BID), combination BLU554 plus fenofibrate, or combination BLU554 plus gemfibrozil (30 mg/kg QD, 100 mg/kg QD, 150 mg/kg QD, or 300 mg/kg QD) for 6 days. Before the final day of the study, mice were fasted overnight and serum was collected 4 hours after the final dose for C4 analysis. The vehicle for fenofibrate and gemfibrozil was 0.5% methylcellulose/0.5% tween 80, and the vehicle for BLU554 was 80% PEG400/4% hydroxypropyl-β-cyclodextrin. In the combination treatment groups, the compounds were formulated and dosed separately. Columns represent mean C4 value (n=3-4 per group) and error bars represent standard error of the mean. Serum C4 (7-alpha-hydroxy-4-cholestene-3-one) analyses were carried out on an Agilent 6495 triple quadrupole mass spectrometer with jet stream source coupled to an Agilent 1290 UPLC stack. Data was processed using Waters TargetLynx data processing software. Calibration curves were generated using reference standard (7A4C; Sigma-Aldrich). Calibration range was from 0.5ng/ml-200 ng/ml with an internal deuterated standard (7A4C-D7; Avanti Polar Lipids) concentration of 100 ng/ml.

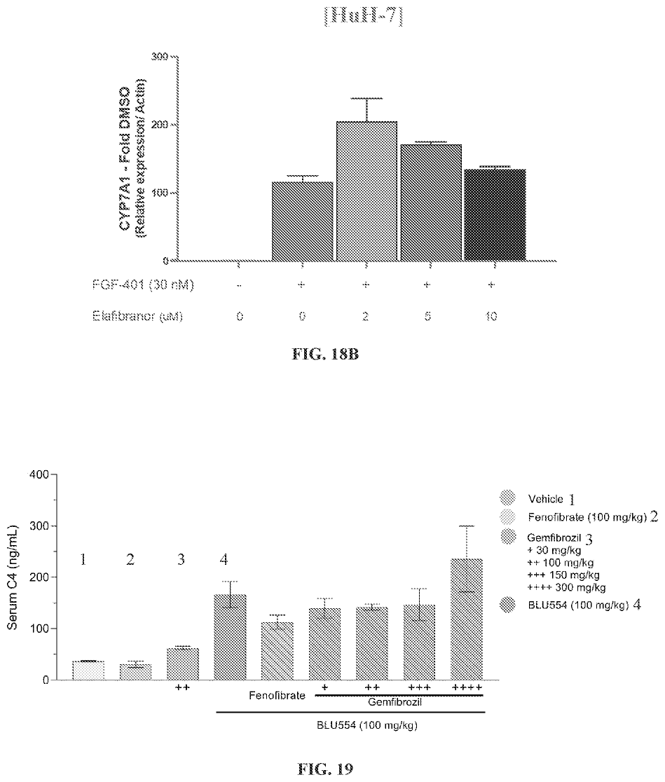

The results are shown in FIG. 19.

1. A method of treating a subject in need of an anti-fibroblast growth factor receptor 4 (anti-FGFR4) therapy comprising administering to the subject a therapeutically effective amount of a peroxisome proliferator-activated receptor (PPAR) alpha agonist in combination with the anti-FGFR4 therapy.

2. The method of claim 1, wherein the anti-FGFR4 therapy is an FGFR4 inhibitor comprising a small molecule FGFR4 inhibitor, an anti-FGFR4 antibody or a binding fragment thereof, an anti-FGF19 antibody or a binding fragment thereof, or an anti-klotho beta antibody or a binding fragment thereof.

3. The method of claim 2, wherein the small molecule FGFR4 inhibitor is roblitinib (FGF401), H3B-6527, ICP-105, fisogatinib (BLU554), INCB062079, erdafitinib, futibatinib, pemigatinib, infigratinib, or a combination thereof.

4. The method of claim 2, wherein the anti-FGFR4 antibody is a humanized anti-FGFR4antibody or a binding fragment thereof.

5. (canceled)

6. The method of claim 1, wherein the anti-FGFR4therapy comprises a combination of a FGFR4 inhibitor and a second chemotherapeutic agent.

7.-10. (canceled)

11. The method of claim 1, wherein the PPAR-alpha agonist is fenofibrate, fenofibric acid, ciprofibrate, gemfibrozil, bezafibrate, elafibranor, pemafibrate, or a combination thereof.

12. The method of claim , wherein the FGFR4 inhibitor is administered at a dose of between
about 0.5 mg and about 3000 mg daily, or
about 0.01 mg/kg and about 50 mg/kg daily.

13.-14. (canceled)

15. The method of claim 1, wherein the PPAR alpha agonist is administered at a dose of between about 0.05 mg and about 3000 mg daily, or about 0.001 mg/kg and about 50 mg/kg daily.

16. (canceled)

17. The method of claim 1, further comprising administration to the subject a bile acid sequestrant.

18. The method of claim 17, wherein the bile acid sequestrant is cholestyramine, colestipol, colesevelam, or a combination thereof.

19. (canceled)

20. The method of claim 1, wherein the PPAR alpha agonist is administered prior to, simultaneously with, or following administration of the anti-FGFR4 therapy.

21. The method of claim 17, wherein the PPAR alpha agonist and the bile acid sequestrant are administered prior to, simultaneously with, or following administration of the anti-FGFR4 therapy.

22. (canceled)

23. The method of claim 1, wherein the subject is in need of treatment for proliferative disease, metabolic disease, cardiovascular disease, or kidney disease.

24. The method of claim 1, wherein the subject is in need of treatment for a proliferative disease that is an FGFR4-mediated cancer, hepatocellular carcinoma, cholangiocarcinoma, or a solid tumor.

25. The method of claim 1, wherein the subject is in need of treatment for a metabolic disease comprising non-alcoholic steatohepatitis (NASH), diabetes, concentric cariac hypertrophy, cardiovascular disease, chronic kidney disease, or ventricular hypertrophy.

26.-30. (canceled)

31. The method of claim 1, wherein the method provides one or more of: a reduction in the number of anti-FGFR4 therapy-related adverse events, a reduction in the anti-FGFR4 therapy-related adverse event frequency, a reduction in the anti-FGFR4 therapy-related adverse event severity, an increased duration of the anti-FGFR4 therapy, an increased daily dose of the anti-FGFR4 therapy, and an increases patient compliance of the subject to the anti-FGFR4 therapy; wherein the adverse event is diarrhea, nausea, vomiting, increased level of aspartate transaminase (AST), increased level of alanine transaminase (ALT), increased level of gamma-glutamyl transferase (GGT), increased level of serum bilirubin, increased prothrombin time (PT), or a combination thereof.

32.-34. (canceled)

35. A composition comprising a fibroblast growth factor receptor 4 (FGFR4) inhibitor, a peroximsome proliferator-activated receptor (PPAR) alpha agonist, and a pharmaceutically acceptable excipient.

36. (canceled)

37. The composition of claim 35, wherein the FGFR4 inhibitor comprises a small molecule FGFR4 inhibitor that is roblitinib (FGF401), H3B-6527, ICP-105, fisogatinib (BLU554), INCB062079, erdafitinib , futibatinib, pemigatinib, infigratinib, or a combination thereof, an anti-FGFR4 antibody or a binding fragment thereof, an anti-FGF19 antibody or a binding fragment thereof, or an anti-klotho beta antibody or a binding fragment thereof; and the PPAR alpha agonist is fenofibrate, fenofibric acid, ciprofibrate, gemfibrozil, bezafibrate, elafibranor, pemafibrate, or a combination thereof.

38.-44. (canceled)

45. The composition claim 40, wherein the bile acid sequestrant is cholestyramine, colestipol, colesevelam, or a combination thereof.

46.-50. (canceled)
